(19) United States
(12) Patent Application Publication
(10) Pub. No.: US 2004/0230181 A1
(43) Pub. Date: Nov. 18, 2004

(54) Urine pump for condom catheters and method for using

(75) Inventor: C. David Cawood, Houston, TX (US)

Correspondence Address: GARY L. BUSH, ANDREWS KURTH LLP, SUITE 4200, 600 TRAVIS, HOUSTON, TX, 77002, US

(73) Assignee: Cawood Family Limited Partnership

(21) Appl. No.: 10/846,032

(22) Filed: May 14, 2004

Related U.S. Application Data
(60) Provisional application No. 60/471,432, filed on May 16, 2003.

(51) Int. Cl. A61M 1/00; A61M 27/00

(52) U.S. Cl. 604/544

(57) ABSTRACT

An apparatus and method for collecting urine from an incontinent person including a condom catheter fluidly coupled to the inlet of a urine pump, with the outlet of the pump fluidly coupled to a urine collection bag suspended about the person's abdomen by an integral strap. The pump is fastened to the strap adjacent to the urine bag. The pump mechanism has a resilient flexible tubular section that is provided with inlet and outlet check valves and functions by displacing liquid present within the tubular section by physically deforming the tubing. A cam, driven by a solenoid, causes a crimping of the tubular section which expels fluid through pump discharge. When the force causing physical deformation is released, the resilient tubular section returns to its original shape, providing a suction at the pump inlet. For continuous or intermittent pumping, the solenoid is repeatedly actuated and de-energized according to a timing sequence that is user controllable according to expected urine leakage conditions via the electronic control circuitry. When the user wishes to urinate, the user may simply void in the normal fashion, as free flow of urine through pump assembly is permitted. A manual activation switch allows the user to pump accumulating fluid from the condom catheter independently of the timing sequence, if needed.

### CROSS REFERENCE TO RELATED APPLICATION

This application is based upon provisional application 60/471,432 filed on May 16, 2003, the priority of which is claimed.

### BACKGROUND OF THE INVENTION

1. Field of the Invention

This invention relates generally to urine collection devices for persons who suffer from uncontrollable urine leakage and specifically to a urine pump which allows a condom catheter to be used in conjunction with a urine collection container which is disposed above the level of the urethral opening of a user.

2. Description of the Prior Art

There are several hundred thousand people in the U.S. alone who continually wear an internal indwelling urinary drainage catheter, coined a “Foley” catheter after the doctor who invented it. A user continuously wears a Foley catheter for two principle reasons—the inability to void (urinate) and incontinence (leakage) of urine. The Foley catheter is inserted into the bladder through the urethra and is designed to be held in place by a balloon located at the bladder end of the catheter which is inflated after the catheter is in place. A Foley catheter is typically changed every thirty days because mineral salts, normally present in urine, tend to accumulate and obstruct the catheter.

Early medical practice was to locate the drainage bag below the bladder so it could drain by gravity. Commonly, the user would wear a urine collection bag strapped to his or her leg. A common leg bag (10) of prior art is shown in FIG. 1, equipped with a Foley catheter attached thereto (12). The bag has straps (14) for securement around a leg and a drain valve (16). However, the leg bag has significant drawbacks, including difficulty in concealing the bag, difficulty in emptying the bag, and a restriction of normal activities of daily living because of embarrassment from bag bulging, leaking, and difficulty of concealment. More significantly, the leg bag has a tendency to slide down the leg as it gets full. A wearer must tighten the holding straps to keep the bag from falling down the leg, which results in bruising and skin damage. Lastly, the leg bag is not suitable for sleeping because of its limited capacity.

A bedside urine collection bag (20) of prior art is shown in FIG. 2. The bedside bag replaces the leg bag (10) (See FIG. 1) during sleeping or for wheelchair use. The wearer disconnects the distal end of the catheter from the leg bag and connects it to the extra long inlet tubing (22) of the bed bag (20). When the user wishes to have increased mobility, the process is reversed. The bedside bag is designed to hang on the side of a bed with a hook (24). However, this bag is particularly subject to the wearer catching, snagging or stepping on the inlet tubing which can result in the wearer falling or in an inadvertent extraction of the Foley catheter with its balloon inflated, with resulting trauma and bleeding. Extracting the catheter with its balloon inflated can result in sepsis, the dissemination of the blood with bacteria, and occasionally, death.

Catheter-associated bacteriuria is a common infection acquired with use of a Foley catheter/leg bag/bedside bag combination. To help prevent pathogenic bacteriuria, a closed catheter system is recommended, which minimizes exposure to non-native bacteria to which the wearer has no innate resistance. However, opening the drainage system to change from a bedside to leg bag or vice versa violates the accepted standard to keep drainage systems closed to prevent exposure to outside environmental (nonsocomial) foreign organisms, and thus can result in significant infections of the upper urinary tract and bloodstream.

A urine collection bag (30) which is worn over the abdomen and secured around the waist with straps (32), described in U.S. Pat. Nos. 4,449,971 and 6,045,542 issued to Cawood, incorporated in their entirety herein by reference, is shown in FIG. 3. This urine collection bag (30) solves the problem of opening the catheter-collection bag system, because the large capacity belly bag is suitable for both day and night use, allowing the wearer to both comfortably sleep and be active. The belly bag does not subject the wearer to bruising and skin damage as does the leg bag, and it is easier to conceal. FIG. 3 shows the belly bag equipped with a drain valve (34), shown folded in an upward, stowed position behind a strap (36). Because the drain (34), when unfolded, is located near the wearer's urethral and clothing openings, draining the bag is more natural and simplified than draining either a bedside bag or a leg bag. FIG. 3 also shows an inlet (38) equipped with a anti-reflux valve (39) as set forth in U.S. Pat. No. 6,352,526, incorporated herein by reference. The anti-reflux valve (39) prevents urine from flowing out of the bag through the inlet (38). Because of its position, the belly bag also reduces the medical hazards of inadvertent extraction of an inflated Foley catheter and resultant injury.

Because the belly bag is located above the bladder and urethral opening of the wearer, the urine must overcome the force of gravity in order to flow upward into the bag. It has been well documented in the medical literature that the human bladder has an innate resting pressure which is transmitted through an indwelling catheter and is sufficient to drive urine upwards into the belly bag. This resting pressure allows the user to completely drain the bladder against the vertical head of the inlet tubing (13) (See FIG. 1, U.S. Pat. No. 4,449,971) to the belly bag.

Although there are several hundred thousand people in the U.S. alone who continually wear an internal indwelling urinary drainage catheter, or Foley catheter, there is a much larger group of individuals who have leakage/incontinence who do not wear indwelling internal (Foley) catheters. Indeed, most health care workers rightfully believe that Foley drainage should be avoided if there is any other way to manage incontinence. Foley drainage is used for patients when they are unable to tolerate other methods and who do not want or are not candidates for surgery. Common alternatives to Foley catheters include intermittent self-catheterization, wearing diapers, or, for males, wearing an external condom catheter.

Many men have uncontrollable voiding, leakage or dribbling of urine which can be drained into a urine collection bag by use of a condom catheter. A condom catheter consists of a condom-style latex or plastic sheath with a tubular extension on the end such that it can be attached to a drainage bag. It is attached to the penis with an adhesive that is part of the sheath's interior or secured by external compressive devices such as elastic tape, Velcro, et cetera. Because of a number of factors, it is recommended that the wearer remove and reply a new condom sheath daily.

Because a condom catheter is located external to the bladder, there is no inherent bladder resting pressure exerted to cause the urine to flow upwards. The condom catheter is used with collection bags which are located lower than the urethral outlet of the wearer. If a condom catheter is used to propel urine upwards to a belly bag, urine will accumulate in the condom and tubing leading to the bag, resulting in possible infection, maceration, erosion, ulceration and necrosis (tissue death) of the penile tissues. Thus, typically, the condom sheath is connected to a bedside bag at night and to a leg bag worn on the thigh or lower leg during the day. However, many of the same problems inherent with the bedside and leg bags used with Foley catheters, described above, apply to these bags when used with the condom catheter: Wearers trip on bedside bag tubing, the leg bag bruises tissue and damages skin, and the bags negatively affect an active lifestyle. Additionally, since many wearers have limited mobility from paralysis, stroke, etc., the difficulties of draining these bags pose a very troublesome problem.

It is desirable, therefore, to allow use of the belly bag in conjunction with a condom catheter.

### IDENTIFICATION OF OBJECTS OF THE INVENTION

A primary object of the invention is to provide a system and method to allow wearers of condom catheters to use an abdominally worn urine collection bag by providing a urine pump designed and arranged to be worn by the user.

Another object of the invention is to provide a system and method to allow such patients a freedom of movement, ease of concealment, and ease of drainage not afforded by standard gravity drainage bags such as the commonly used leg bag and bedside bag.

Another object of the invention is to provide a urine pump which is self-priming.

Another object of the invention is to provide a urine pump which has an adjustable rate of pumping, as well as a means to override the pump with an “on demand” button. The preferred duty cycle is 2 seconds/minute on (pumping), 58 seconds/minute off (open). This would be patient adjustable depending on his individual needs between 1 and 15 seconds on (pumping), and the remainder of the 60 seconds off (open).

Another object of the invention is to provide a urine pump which is lightweight and inexpensive. Although the pump may include an osmotic pump, a roller pump, solenoids or linear actuators, for the size, cost and ease of use a simple solenoid or roller actuator is preferred.

Another object of the invention is to isolate the urine from the pumping mechanism so that the mechanism will not be subject to the corrosive salts inherent in urine.

Another object of the invention is to provide a urine collection system which uses inexpensive and replaceable tubing and valves which last for 30 days, which are user friendly, and which are easy to replace.

Another object of the invention is to provide a urine collection system which allows the free flow of urine to the collection bag between pump cycles so that a normal voiding episode can occur.

Another object of the invention is to provide a urine collection system which has a self-contained power supply. The system preferably uses batteries, or a rechargeable battery pack which is easily snapped in place.

### SUMMARY OF THE INVENTION

The objects identified above, as well as other features and advantages of the invention are incorporated in an apparatus for collecting urine from a person with incontinence/urine leakage, the apparatus including a condom catheter fluidly coupled via a urine collection tube to the inlet of a urine pump assembly, with the outlet of the urine pump assembly fluidly coupled via a flexible urine inlet tube to the inlet of a urine collection bag suspended about the person's abdomen by an integral strap. The urine pump is clipped or otherwise fastened to the strap adjacent to urine bag. The urine bag includes a drain assembly with a drain tube, which may be of any suitable length. A manually controlled drain valve permits the user to drain collected urine from the collection bag.

The condom catheter consists of a condom-style latex or plastic sheath which is removably coupled to a urine collection tube. The condom catheter is adapted to receive a portion of the penis using an adhesive which is coated to the interior surfaces of sheath, or other suitable means. Other types of urine collection devices may be provided for use by females to transfer urine from the female to the urine collection tube.

The urine collection tube is connected to the inlet of a urine pump mechanism. The pump mechanism has a resilient flexible tubular section made of an elastomeric or polymer material that has shape memory. The resilient tube is provided with inlet and outlet one-way check valves. The pump mechanism functions by displacing liquid present within the resilient tubular section by physically deforming the tubing. The displacement acts in concert with the check valves which permit only unidirectional movement of fluid through the flexible tubular section. A cam or other deforming object, driven by a solenoid or other means, causes a crimping of the tubular section which expels fluid through the pump, through the outlet check valve. When the force causing physical deformation is released, the elastic memory of the resilient material from which the tubular section is composed causes the tubular section to return essentially to its original shape, providing a suction to the pump inlet check valve. The outlet one-way check valve is connected to the flexible urine inlet tube of the belly bag.

The intermediate tubular section of the pumping mechanism is located within a channel between two flanges and is releasably secured in place by a keeper member. The urine collection system incorporates interchangeable components that are easily replaced by the user to enable the system to be operable at all times. The keeper member is simply removed, thereby releasing the check valves and intermediate tubular section. New tubing and valves are then connected between the inlet connection of the bag and a new condom catheter, and the keeper is replaced.

The solenoid or other actuating mechanism is selectively energized by an electrical circuit having a source of electrical energy, such as a battery pack or individual cells, located within an electronics housing. When the actuator is energized, it moves the cam toward the intermediate tubular section and compresses it. When the actuator is de-energized, it retracts the cam away from the tubular section, permitting the tubular section to expand.

For continuous or intermittent pumping, the solenoid or other actuating means is repeatedly energized and de-energized according to a controllable timing sequence. For automated pumping control, a timer device is included to achieve periodic pump actuation according to a timing sequence matched to the wearer's urine leakage rate. The timer device is user adjustable, permitting the user to manually change the pumping sequence to accommodate changes in urine leakage rate. When the user wishes to urinate, the user may simply void in the normal fashion. When the pump solenoid is de-energized, thereby removing the compressive force from the tubing, free flow of urine through pump assembly is permitted.

### BRIEF DESCRIPTION OF THE DRAWINGS

The invention is described in detail hereinafter on the basis of the embodiment represented in the accompanying figures, in which:

FIG. 1 is a perspective illustration of a flexible urine collection bag of prior art designed to be worn on a user's leg;

FIG. 2 is a perspective illustration of a flexible urine collection bag of prior art designed to be hung on a bedside while a user sleeps;

FIG. 3 is a perspective illustration of a flexible urine collection bag of prior art designed to worn around a user's abdomen;

FIG. 4 is a perspective illustration of a user wearing the urine collection system according to the invention, with a urine collection bag being supported at the waist of the user and with the inlet opening of the urine collection bag being located well above the urine discharge of the user;

FIG. 5 is a perspective illustration of the urine collection system according to the invention, illustrating a condom catheter for urine collection, a flexible belly bag for urine collection, and an electrically operated pump mechanism for pumping urine from the condom catheter into the flexible urine collection bag;

FIG. 6 is a detailed perspective illustration of the tubular pump and check valve assembly of FIG. 5 being prepared by removal of a keeper element to permit periodic replacement by the user as needed to maintain patency of the system;

FIG. 7 is a perspective illustration showing check valve assembly and the urine pump mechanism of FIG. 5 in detail, with the electronics revealed and with the cam (pump mechanism) being shown in an open or relaxed position, with the pump allowing free one-way flow;

FIG. 8 is a detailed perspective illustration of the check valve assembly and the urine pump mechanism of FIG. 5, showing the cam (pump mechanism) in a pumping or displaced position; and

FIG. 9 is an electronic schematic circuit diagram showing the pump control circuitry of the present invention in detail.

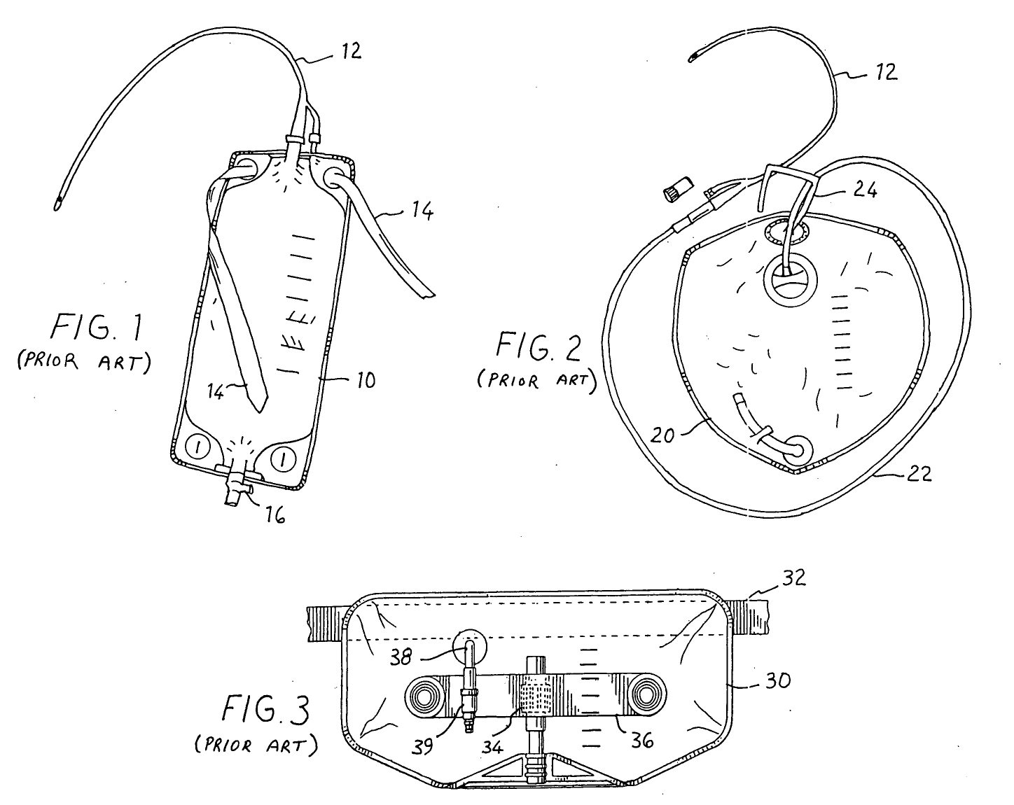

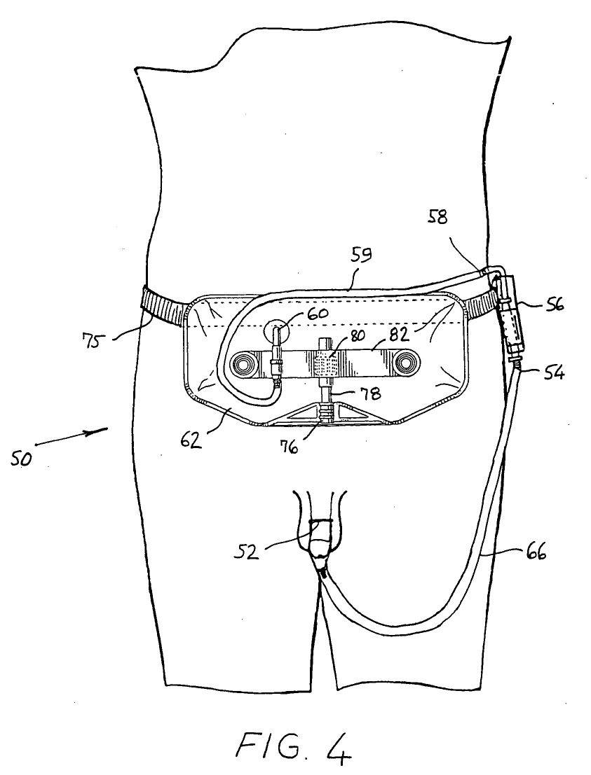

### DESCRIPTION OF THE PREFERRED EMBODIMENT OF THE INVENTION

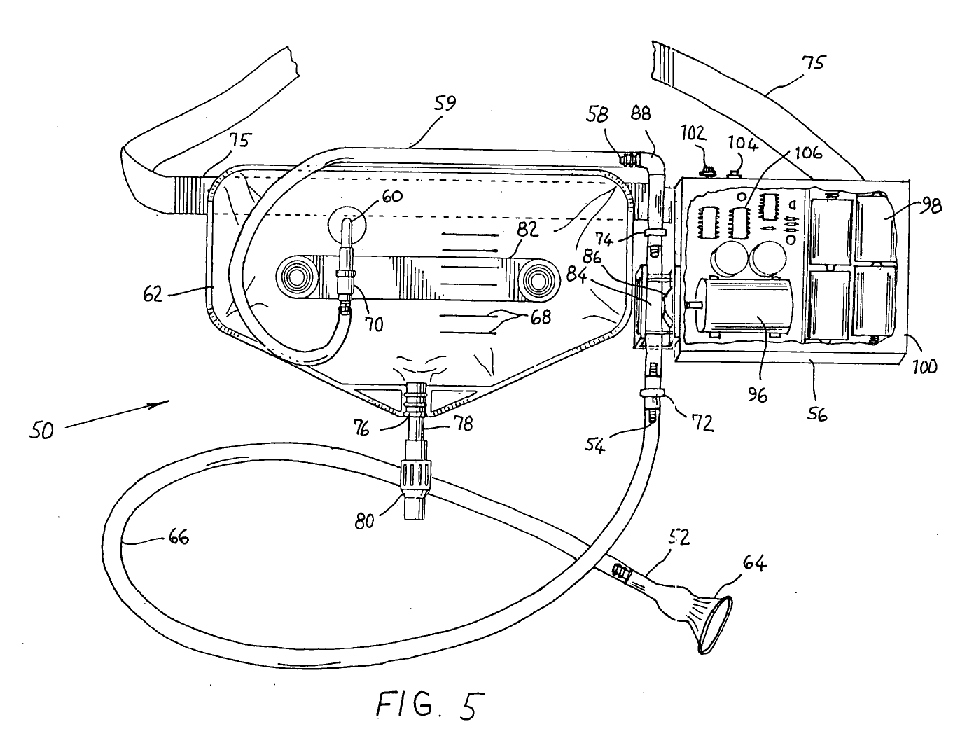

FIG. 4 illustrates the preferred embodiment according to the invention. The urine collection system 50 includes a condom catheter 52 fluidly coupled via a urine collection tube 66 to the inlet 54 of a urine pump assembly 56, with the outlet 58 of the urine pump assembly fluidly coupled via a flexible urine inlet tube 59 to the inlet 60 of a urine collection bag 62. The system 50 is shown on a male having urine leakage/incontinence with urine bag 62 suspended about his abdomen by an integral strap 75. Urine pump 56 is clipped or otherwise fastened to the strap 75 at the wearer's side, although it may be placed immediately adjacent to urine bag 62 at the wearer's front. Condom catheter 52 is attached to the man's penis to capture urine leakage, which is pumped into urine bag 62. The urine bag 62 includes a drain fitting 76, to which is connected a drain tube 78, which may be of any suitable length. A manually controlled drain valve 80 is provided at the distal end of the drain tube 78, thus permitting the user to drain collected urine from the collection bag 62 into a toilet or other receptacle for disposal. The drain fitting 76, drain tube 78, and drain valve 80 may be folded upwards and stowed under strap 82, as shown.

A more detailed illustration of the urine collection system 50 of FIG. 4 is shown in FIG. 5. The pump assembly 56 is shown with a portion of its cover cut away to expose the electronic components therein. The drain fitting 76, drain tube 78, and drain valve 80 are shown in an unfolded position which is used to drain the urine collection bag 62.

Referring to FIG. 5, the condom catheter 52 consists of a condom-style latex or plastic sheath 64 which is removably coupled to a urine collection tube 66. The condom catheter is adapted to receive a portion of a penis using an adhesive which is coated to the interior surfaces of sheath 64 or other suitable means. Other types of urine collection devices may be provided for female use to transfer urine from a female to the urine collection tube 66.

The urine collection bag 62 is preferably a belly bag constructed in accordance with U.S. Pat. No. 6,045,542, although other urine collection bags may be used. The fluid-tight, flexible bag has an inlet 60 located near the top of the bag, through which urine is introduced into the bag. Preferably, the bag is constructed from a transparent or translucent material, so that the fluid level within is easily measured against indicia 68. FIG. 4 shows an optional anti-reflux valve 70 installed at inlet 60. The anti-reflux valve, which prevents urine from flowing out of the bag 62 through inlet 60, is not necessary because urine pump assembly 56 includes check valves 72, 74 which prevent back flow. If installed, however, the anti-reflux valve is preferably constructed in accordance with U.S. Pat. No. 6,352,526. The urine collection bag includes an integral strap assembly 75 which allows the bag to be suspended around the waist of a wearer.

Referring to FIG. 5, the urine collection tube 66 is connected to the inlet 54 of a urine pump mechanism 56. The urine pump mechanism has a resilient, flexible intermediate tubular section 84 that is provided with inlet and outlet one-way check valves 72 and 74. The urine pump mechanism 56 functions by displacing liquid present within the tubular section 84 by physically deforming the tubing. The displacement acts in concert with the check valves which permit only unidirectional movement of fluid through the tubular section 84. A cam element 86 or other deforming object, driven by a solenoid 96 or other actuator, causes a crimping of the tubular section 84 which expels fluid through pump discharge 58. When the force causing physical deformation is released, the elastic memory of the resilient material from which the intermediate tubular section is composed causes the tubular section 84 to return essentially to its original shape, providing a suction at the pump inlet 54. The flexible tubular section 84 is typically composed of a polymer material that minimizes the build-up of salts from urine, but regardless of the material from which it is composed, it has an elastic memory causing it to return to its original tubular shape in absence of a deforming object. The outlet one-way check valve 74 is provided with an elbow fitting 88 which is connected with the flexible urine inlet tube 59.

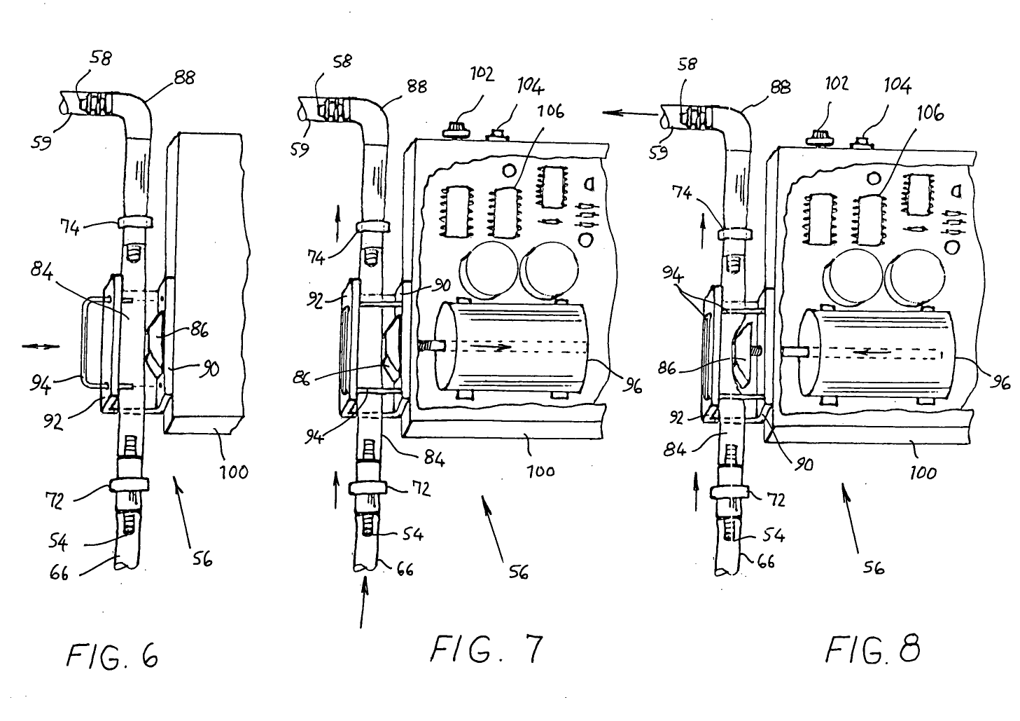

Referring to FIG. 6, the intermediate tubular section 84 of the pumping mechanism 56 is located within a channel between channel plates or flanges 90 and 92 and is releasably secured in place by a keeper member 94. The estimated life of the tubing and check valves is about 30 days, after which time the internal surfaces will have accumulated deposits of mineral salts from the urine that is continuously present therein which can obstruct the flow path. Periodic replacement of the flexible tubing and check valves assembly is also important to ensure that an accumulation of non-native bacteria does not occur in the urinary system of the patient. Thus, the urine collection system incorporates interchangeable components that are easily replaced by the user to enable the system to be operable at all times. The keeper member 94 is simply removed as shown in FIG. 6, thereby releasing the intermediate tubular section 84 and check valves 72, 74. New tubing and valves are then connected with the inlet connection 60 (See FIG. 5) of the bag and a new condom catheter 52 (See FIG. 5), and the keeper 94 is replaced, thereby securing the assembly into the pumping channel. Preferably, the valves and tubing will be furnished as a pre-assembled set in sterile packaging so that the entire tubing assembly, from the urine collection bag to the user's collecting device (condom catheter or similar), can be replaced in one simple operation.

FIGS. 7 and 8 illustrate the pumping action of urine pump assembly 56 in greater detail. The resilient tubular section 84 defines an internal chamber who's volume is diminished when the tubular section is collapsed and who's volume is expanded when the tubular section expands. As shown in FIG. 7, when the tubular section expands, a negative pressure condition (suction) is developed within the internal chamber which opens the inlet check valve 72, shuts the outlet check valve 74, and serves to draw urine from the urine collection tube 66. As shown in FIG. 8, to displace urine that is present within the internal chamber, a cam element 86 is forced against the tubular section 84 and deforms or compresses the flexible intermediate tubular section, applying pressure to the urine therein. During the compression stroke, inlet valve 72 is forced shut and outlet valve 74 is forced open to allow urine flow from the pump discharge 58 through inlet tubing 59 into urine bag 62. The pumping cycle begins again when the cam element 86 is retracted, with the natural expansion of the intermediate tubular section 84 creating a suction. This expansion and contraction activity is electronically controlled to cause a pumping activity, with urine being caused to flow from the wearer's collection device into a urine collection bag 62. For continuous or intermittent pumping, the solenoid or other compressive means is repeatedly actuated and de-energized according to a timing sequence that is controllable according to expected urine leakage conditions via the electronic control circuitry.

As shown in FIG. 7, when the user wishes to urinate, the user may simply void in the normal fashion. When the pump solenoid 96 is de-energized, free flow of urine through pump assembly 56 is permitted. Very little pressure is needed to open the inlet and outlet check valves 72, 74 to permit unobstructed flow of urine through the pump mechanism to the urine collection bag 62 (See FIG. 5). The check valves preferably have an unseating pressure of 0.2 P.S.I. or less. Thus, it is not necessary for the user to preset or override the pumping system when normal voiding is desired, because the bladder can develop sufficient pressure to force urine upwards into the bag. Any urine remaining at the penis after normal urination is pumped away using pump 56 in order to avoid adverse affects of urine accumulation around the penis.

Referring back to FIG. 5, the solenoid 96 or other actuating means is selectively energized by an electrical circuit having a source of electrical energy, such as a battery pack or individual cells 98 located within an electronics housing 100. When the solenoid or other actuating means is energized, the cam element 86 is moved toward the intermediate tubular section 84 and compresses it. When the solenoid is de-energized, the cam element 86 is retracted and the intermediate tubular section 84 is permitted to expand.

For automated pumping control, a timer device 106 is located within the electronics housing 100 to achieve periodic pump actuation according to a timing sequence matched to the wearer's urine leakage rate. Preferrably, the timer device is user adjustable using a potentiometer, switch or other mechanism 102, permitting the user to manually change the pumping sequence to accommodate changes in urine leakage rate. The pump rate is normally functional at between 2 and 30 cycles per minute and the “on” time of the pumping system is one second or less. An override switch 104 is also connected within the electronic circuitry and permits the user to manually override the timer circuit 106 of the pump to manually activate the pumping. This function is used when the patient senses the presence of urine at the penis to pump away the undesired accumulation of urine and then continues in the selected timed mode as set/adjusted by the user.

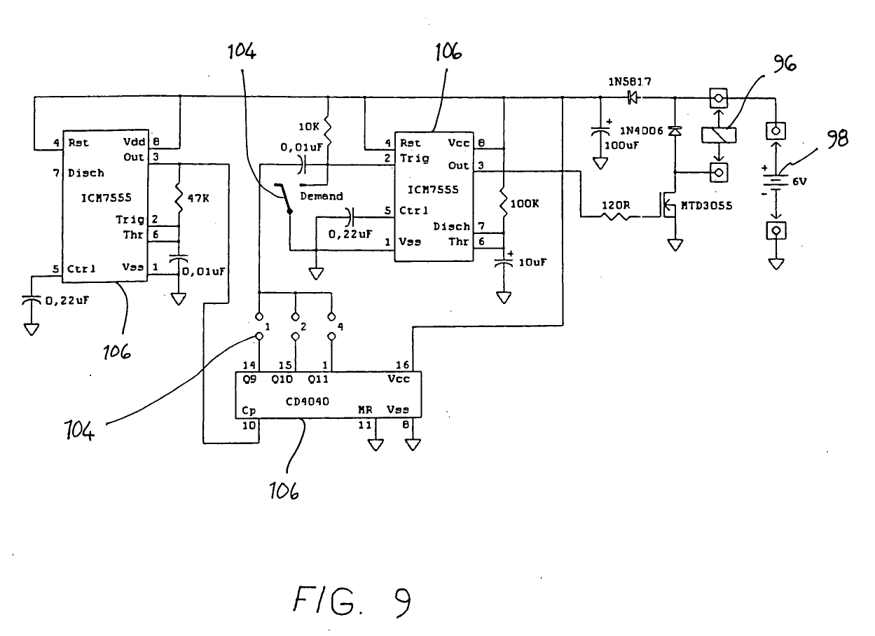

FIG. 9 is a detailed schematic diagram of the electronic control circuitry of pump assembly 56 according to the invention. Battery 98, solenoid 96, manual demand switch 102, adjustable rate switch 104, and timer circuit 106 are shown.

Though the urine collection system of the present invention is designed particularly for collection of urine from male patients using condom type catheters, the invention is applicable for use by female patients as well, assuming the provision of a suitable urine collector for the female anatomy. Further, the present invention may have effective application in other medical and non-medical disciplines. Though the pump system of the present invention is designed particularly for electrical operation and control, pumps which are powered or controlled by other means are within the spirit and scope of the present invention.

While the preferred embodiment of the invention has been illustrated in detail, modifications and adaptations may occur to those skilled in the art. Such modifications and adaptations are in the spirit and scope of the invention as set forth in the following claims:

What is claimed is:

1. A urine collection apparatus (50) comprising,
a collector (52) designed and arranged to fit against a person and receive urine discharge therefrom,
a pump apparatus (56) having an inlet (54) and an outlet (58) and being designed and arranged to periodically and automatically pump fluid from said inlet to said outlet, said collector fluidly coupled to said inlet, and
a container (62) fluidly coupled to said outlet, wherein
said urine collection system receives said urine discharge from said person and automatically pumps said urine discharge to said container.

2. The urine collection apparatus (50) of claim 1 wherein,
said container is a flexible urine collection bag (62) having an input (60) and a drain (76), said input fluidly coupled to said outlet (58) with a flexible tube (59).

3. The urine collection apparatus (50) of claim 2 wherein,
said urine collection bag (62) includes a strap(75) for securement of the bag about said person.

4. The urine collection apparatus (50) of claim 3 wherein,
said urine collection bag (62) is designed and arranged to be worn about an abdomen of said person.

5. The urine collection apparatus (50) of claim 1 wherein,
said collector is a condom catheter (52) adapted to receive a portion of a penis of said person and attach thereto.

6. The urine collection apparatus (50) of claim 5 wherein
said condom catheter (52) is fluidly coupled to said inlet (54) with a flexible tube (66).

7. The urine collection apparatus (50) of claim 1 wherein said pump apparatus (56) comprises,
a resilient tube (84),
a displacing member (86) disposed generally adjacent to said resilient tube, having an engaged position wherein said displacing member (86) compresses said resilient tube (84), and having a disengaged position wherein said displacing member (86) does not compress said resilient tube (84),
an actuator (96) coupled to said displacing member (86) and designed and arranged to move said displacing member (86) from said disengaged position to said engaged position and vice versa,
an inlet check valve (72) coupled between a first end of said resilient tube (84) and said collector (52), said inlet check valve defining said inlet (54) of said pump apparatus (56) and oriented to allow flow only from said collector (52) to said resilient tube (84), and
an outlet check valve (76) coupled between a second end of said resilient tube (84) and said container (62), said outlet check valve defining said outlet (58) of said pump apparatus (56) and oriented to allow flow only from said resilient tube (84) to said container (62).

8. The urine collection apparatus (50) of claim 7 wherein,
said actuator (96) is a linear actuator.

9. The urine collection apparatus (50) of claim 8 wherein,
said actuator (96) is a solenoid.

10. The urine collection apparatus (50) of claim 8 wherein,
said actuator (96) is a roller.

11. The urine collection apparatus (50) of claim 7 wherein,
fluid can freely flow through said inlet check valve (72), through said resilient tube (84) and through said outlet check valve (74) when said displacing member (86) is in said disengaged position.

12. The urine collection apparatus (50) of claim 1 wherein said pump apparatus comprises,
a battery (98) for powering said pump apparatus (56).

13. The urine collection apparatus (50) of claim 1 wherein
said pump apparatus (56) is designed and arranged to fasten to a strap (75) worn around said person.

14. The urine collection apparatus (50) of claim 1 wherein said pump apparatus (56) comprises,
a timer (106) designed and arranged to activate said pump apparatus (56) to pump periodically, with a period which can be adjusted by said person.

15. The urine collection apparatus (50) of claim 1 wherein said pump apparatus (56) comprises,
an activation device (104) wherein said person can manually cause said pump apparatus (56) to pump.

16. The urine collection apparatus (50) of claim 7 wherein,
said collector (52) is fluidly coupled to said inlet (54) with a collection tube (66),
said container (62) is fluidly coupled to said outlet (58) with an inlet tube (59), and
said collection tube (66), said inlet check valve (72), said resilient tube (84), said outlet check valve (74) and said inlet tube (59) are replaceable as a single unit.

17. A urine pump (56) comprising,
a resilient chamber including and bounded by a resilient body (84), an inlet check valve (72) coupled to a first end of said body, and an outlet check valve (74) coupled to a second end of said body, said resilient chamber characterized by having an original shape, and
a displacement member (86) designed to periodically, automatically and compressively deform said resilient chamber to displace a portion of liquid therein through said outlet check valve (74) and to relax to allow said resilient chamber to return to said original shape and suck a liquid through said inlet check valve (72) into said resilient chamber.

18. The urine pump (56) of claim 17 further comprising,
an actuator (96) mechanically coupled to said displacement member (86), said actuator having an energized state which causes said displacement member (86) to compressively deform said resilient chamber and a de-energized state which causes said displacement member (86) to relax and allow said resilient chamber to be in said original shape.

19. The urine pump (56) of claim 18 further comprising,
a timer (106) electrically coupled to said actuator (96), said timer designed and arranged to periodically and automatically energize said actuator.

20. The urine pump (56) of claim 19 further comprising,
an adjustment device (102) coupled to said timer (106) and designed and arranged to vary a periodicity at which said timer (106) automatically energizes said actuator (96).

21. The urine pump (56) of claim 18 further comprising,
a manual activation device (102) coupled to said actuator (96) which is designed and arranged to cycle said actuator between said energized and said de-energized states.

22. The urine pump (56) of claim 18 further comprising,
a battery (98) coupled to said actuator (96) and designed and arranged to power said actuator.

23. The urine pump (56) of claim 19 further comprising,
a battery (98) coupled to said timer (106) and designed and arranged to power said timer.

24. The urine pump (56) of claim 17 wherein,
said inlet check valve (72) is fluidly coupled to a condom catheter (52), and said outlet check valve (74) is fluidly coupled to a urine collection bag (62).

25. The urine pump (56) of claim 17 wherein,
said resilient chamber (72/84/74) is replaceable as a single unit.

26. A method of collecting urine from a person comprising the steps of,
coupling a first end of a catheter (58) to a urethral opening of said person,
coupling a second end of said catheter to an inlet (54) of a urine pump (56),
coupling an outlet (58) of said urine pump to an input (60) of a urine collection container (62), and
causing said urine pump (56) to periodically and automatically transfer urine in said catheter (52) to said urine collection container (62).

27. The method of claim 26 further comprising the step of,
adjusting a pumping rate of said urine pump (56) to match a urine leakage rate of said person.

28. The method of claim 26 further comprising the step of,
manually causing said urine pump (56) to transfer said urine in said catheter (52) to said urine collection container (62).

29. The method of claim 26 further comprising the step of,
with said urine pump (56) in an idle mode, said person urinating through said catheter (52), through said urine pump (56), and into said urine collection container (62).

30. The method of claim 26 further comprising the step of,
disposing said urine collection container (62) at an elevation higher than said urethral opening of said person.

31. The method of claim 26 further comprising the step of,
suspending said urine collection container (62) around the abdomen of said person.

32. The method of claim 26 wherein,
said catheter (52) is a condom catheter designed and arranged to receive a portion of a penis of said person.

33. The method of claim 26 wherein,
said urine is transferred from said catheter (52) to said urine collection container (62) by tubing (66, 59) and valves (72, 74),
said method further comprising the step of replacing said tubing and valves as a combined unit.

34. The method of claim 26 further comprising the step of,
replacing a battery (98) of said urine pump (56).

35. The method of claim 26 further comprising the step of,
recharging a battery (98) of said urine pump (56).

36. The method of claim 26 wherein,
said urine collection container (62) is a flexible urine collection bag.
